(19) United States
(12) Patent Application Publication
(10) Pub. No.: US 2018/0008827 A1
(43) Pub. Date: Jan. 11, 2018

(54) DIAGNOSIS AND TREATMENT OF CHRONIC PAIN

(71) Applicant: NIBS NEUROSCIENCE TECHNOLOGIES LTD., Zur Moshe (IL)

(72) Inventors: Iftach DOLEV, Zur Moshe (IL); Hilla FOGEL, Beit Dagan (IL)

(21) Appl. No.: 15/547,505

(22) PCT Filed: Feb. 3, 2016
(86) PCT No.: PCT/IL2016/050123

§ 371 Date: Jul. 30, 2017

Related U.S. Application Data
(60) Provisional application No. 62/111,125, filed on Feb. 3, 2015.

(51) Int. Cl. A61N 1/36; A61N 1/04; A61B 5/0484; A61B 5/04; A61B 5/05; A61N 1/20; A61B 5/00

(52) U.S. Cl. CPC A61N 1/36021; A61N 1/20; A61N 1/0456; A61B 5/4836; A61B 5/04001; A61B 5/05; A61B 5/0484; A61B 5/4824

(57) ABSTRACT

Methods, systems and devices for treating chronic pain by inducing stimulation to a predetermined region of the brain and measuring neural activity response to the stimulation and evaluating a neuroplasticity and/or excitability of neural-structures in a predetermined brain region, then stimulating the predetermined brain region to treat the chronic pain.

### TECHNICAL FIELD

The present disclosure generally relates to the field of pain management.

### BACKGROUND

Pain, when suffered over a long period of time can be categorized as chronic pain, generally, if the time since onset is more than 3-6 months. Chronic pain can originate from the body of a subject, the brain or the spinal cord, and is classified as either neuropathic pain, caused by a condition/malfunction of the nervous system, or nociceptive pain, caused by inflammation or damage to a tissue that activates pain sensory neurons called nociceptors.

Commonly, chronic pain is managed by pain relief drugs such as opioids (morphine, codeine and others), or non-opioid medications (tricyclic antidepressants, serotonin-norepinephrine reuptake inhibitors, anticonvulsants and others). Evidently, the efficacy of the medications varies and may not always help in managing the pain suffered in some cases. Additionally, some medications are associated with harmful side effects including hypogonadism, infertility, impaired immune system, falls and fractures in older adults, neonatal abstinence syndrome, heart problems, sleep-disordered breathing, opioid-induced hyperalgesia, physical dependence, addiction, and overdose.

There is thus a need in the art for effective non-pharmaceutical solutions for chronic pain management.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

Chronic pain has been found associated with affecting the brain structure and function, which are expressed in abnormal anatomical and functional connectivity in various brain regions/areas related to the processing of pain, in addition to causing grey matter loss. These changes in the brain are explained by a neurological/neuro-structural property called neuroplasticity.

Neuroplasticity is manifested in two directions of neural behavior: potentiation, which is when a neural response is increased over time (training) by parts and strengthening of neuronal connection, or depression, which is when a neural response is decreased over time by parts and weakening of neuronal connections

According to some embodiments, there are provided herein devices, systems and methods for treatment/management of chronic pain conditions by stimulating predetermined brain regions of a subject associated with pain, measuring a neural activity response to the stimulation, and assessing the plasticity of the neurons/neural-networks (neuroplasticity) in the predetermined brain regions based on the characteristics of the neural activity response to the stimulation.

According to some embodiments, a treatment may be provided to the subject by providing electrical stimulation sessions targeting the predetermined brain regions, and assessing the effectiveness of the treatment by evaluating the plasticity of the neurons/neural-networks by measuring the neural activity response to neural stimulation.

According to some embodiments, characteristics and parameters of the electrical stimulation sessions may be changed and adapted based on the assessed effectiveness of previous treatment sessions.

According to some embodiments, there is provided a method for treating chronic pain of a subject, the method including inducing a diagnostic stimulation to a predetermined brain region of the subject, measuring a neural activity response to the diagnostic stimulation, evaluating a neuroplasticity and/or excitability of neural-structures in the predetermined brain regions based on the neural activity response to the diagnostic stimulation, and inducing a treatment stimulation to the predetermined brain region based on the evaluated neuroplasticity of the neural structures in the predetermined brain region.

According to some embodiments, the chronic pain is a neuropathic pain. According to some embodiments, the predetermined brain region is selected from a group including: primary motor cortex and primary somatosensory cortex, prefrontal cortex and insular cortex. According to some embodiments, inducing a diagnostic stimulation to a predetermined brain region of the subject includes utilizing transcranial magnetic stimulation (TMS).

According to some embodiments, measuring a neural activity response to the diagnostic stimulation includes obtaining electroencephalogram (EEG) measurements indicative of a neural activity of the brain of the user during and/or after inducing the diagnostic stimulation. According to some embodiments, inducing a treatment stimulation to the predetermined brain region includes electric brain stimulation. According to some embodiments, the inducing electric brain stimulation includes placing a first electrode and a second electrode on the subject, such that an electric signal passing through the user via the first electrode and the second electrode reaches a predetermined brain region.

According to some embodiments, the method further includes evaluating specific brain areas for plasticity changes by dipole fitting the EEG measurements and performing a source modeling for detecting the location (source localized areas) of the specific areas and providing treatment stimulation to the source localized areas.

According to some embodiments, the electric brain stimulation includes transcranial direct current stimulation (tDCS). According to some embodiments, the tDCS utilizes a current density of no more than 5 A/m², and has a duration of no more than 30 minutes. According to some embodiments, the tDCS stimulation is selected to be either Anodal stimulation or Cathodal stimulation for a brain region based on the evaluated neuroplasticity and/or excitability of neural-structures in the predetermined brain regions.

According to some embodiments, the electric brain stimulation includes transcranial alternating current stimulation (tACS). According to some embodiments, the tACS is performed by inducing an alternating current at a frequency in the range of 1-120 Hz at current density of 5 A/m² to be matched with a desired oscillatory behavior needed.

According to some embodiments, the method further includes evaluating a subjective pre-treatment-pain level of the subject before inducing a treatment stimulation, a subjective post-treatment-pain level of the subject after inducing a treatment stimulation, and determining an efficacy of the treatment stimulation based on the difference between the pre-treatment-pain level and the post-treatment-pain level.

According to some embodiments, the method further includes inducing a post-treatment diagnostic stimulation to the predetermined brain region of the subject, measuring a neural activity response to the post-treatment diagnostic stimulation, evaluating a post-treatment neuroplasticity and/or excitability of neural-structures in the predetermined brain regions based on the neural activity response to the post-treatment diagnostic stimulation, and inducing a treatment stimulation to the predetermined brain region based on the evaluated post-treatment neuroplasticity of the neural structures in the predetermined brain region.

According to some embodiments, there is provided a system for treating chronic pain of a subject, the system including a directed inspective/diagnostic stimulation unit, configured to induce a diagnostic stimulation to a predetermined brain region of the subject. a brain activity sensor, configured to measure a neural activity response to the diagnostic stimulation induced by the directed brain stimulation unit, a processing circuitry in communication with the brain activity sensor, the processing circuitry is configured to evaluate a neuroplasticity and/or excitability of neural-structures in the predetermined brain region based on the neural activity response to the diagnostic stimulation, and an electric current stimulation unit including a first electrode and a second electrode, the electric current stimulation unit is configured to induce a treatment stimulation to the predetermined brain region based on the evaluated neuroplasticity of the neural structures in the predetermined brain region, by driving a current signal through the user via the first electrode and the second electrode such that the current reaches the predetermined brain region.

According to some embodiments, the directed inspective stimulation unit includes a transcranial magnetic stimulation (TMS) unit. According to some embodiments, the brain activity sensor includes an electroencephalography (EEG) device. According to some embodiments, the processing circuitry is configured to obtain an EEG signal from the EEG device and evaluate a neuroplasticity and/or excitability of neural-structures in the predetermined brain region by comparing the neural activity response to the diagnostic stimulation with previously obtained neural activity response to previous diagnostic stimulation.

According to some embodiments, the processing circuitry is further configured to estimate locations of the predetermined brain regions based on the obtained EEG signal.

According to some embodiments, the electric current stimulation unit includes a transcranial direct current stimulation (tDCS) device subject. According to some embodiments, the electric current stimulation unit includes a transcranial alternate current stimulation (tACS) device.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a setting of treating chronic pain, according to some embodiments;

FIG. 2a schematically illustrates a repetitive evaluation process, according to some embodiments;

FIG. 2b schematically illustrates a singular evaluation process, according to some embodiments;

FIG. 3 schematically illustrates a session of evaluation and treatment, according to some embodiments;

FIG. 4 schematically illustrates an EEG response to a stimulation burst in a healthy brain, according to some embodiments;

FIG. 5 schematically illustrates a diagnostic stimulation intensity operation range, according to some embodiments;

FIG. 6 schematically illustrates a method for establishing a motor threshold value for evaluation, according to some embodiments;

FIG. 7 schematically illustrates a method for providing diagnostic stimulated EEG response bursts, according to some embodiments;

FIG. 8 schematically illustrates a high-level method for treating chronic pain, according to some embodiments;

FIG. 9 schematically illustrates a subjective method for treating chronic pain, according to some embodiments;

FIG. 10 schematically illustrates an iterative method for treating chronic pain, according to some embodiments;

FIG. 11 schematically illustrates a method for treating chronic pain with tCS stimulation, according to some embodiments;

FIG. 12 schematically illustrates tDCS sessions, according to some embodiments; and

FIG. 13 schematically illustrates a method for treating chronic pain with tDCS stimulation, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Chronic pain has been found associated with affecting the brain structure and function, which are expressed in abnormal anatomical and functional connectivity in various brain regions/areas related to the processing of pain, in addition to causing grey matter loss. These changes in the brain are explained by a neurological/neuro-structural property called neuroplasticity.

Neuroplasticity is manifested in two directions of neural behavior: potentiation, which is when a neural response is increased over time (training) by parts producing strengthening of neuronal connection, or depression, which is when a neural response is decreased over time by parts producing weakening of neuronal connections.

Neuroplasticity can be expressed in both synaptic-neuroplasticity and structural neuroplasticity, and in both, the constructive and destructive directions: synaptic potentiation, synaptic depression, structural potentiation and structural depression.

In synaptic potentiation, the activity of the synapse is increased such that for an action potential, more neurotransmitters are released from the axon terminal into the synapses, or the change may occur in the post synaptic membrane in that the number of neuro-transmitter receptors may increase, or changes to the types of the neurotransmitter receptors, or change in the responses that occur in the second messenger such that the sensitivity to neurotransmitters increases and an increased response per action potential is observed.

On the other hand, synaptic depression can be manifested by a decrease in the neurotransmitter release per action potential, or the number of the receptors is decreased and/or the type of the receptors is changed or changes to second messengers and a decreased response is observed per action potential.

In structural potentiation, the amount, length and strength of dendritic branches and amount and structure of axon terminal forming synapses increases, and the connection between the neurons becomes stronger.

In structural depression, the amount, length and strength of dendritic branches and amount and structure of axon terminal forming synapses increases, and the connection between neurons becomes weaker.

In a normal brain, neuroplasticity comes into effect in that activating a link/connection between neurons commonly promotes potentiation, and the more a connection between neurons is activated, the more the link becomes stronger. On the other hand, not utilizing a link between neurons commonly results in a weakening of the link between the neurons.

In people suffering from chronic pain, the neural structure and the behavior of the neural activity in some pain related brain regions is affected. Therefore, according to some embodiments, the affected brain regions may be detected by measuring a neural activity response to a diagnostic stimulation, and a treatment stimulation may be applied for stimulating and/or inhibiting the neural activity in the affected brain regions for reducing the chronic pain.

According to some embodiments, the affected brain region may include the motor cortex and/or the primary somatosensory cortex (which are stimulated usually by 10/20 electrode positioning of C3, C4, Cz and possibly C5, C6, Cp1, Cp2 corresponding to painful area and or prefrontal cortex (electrodes Fp1, Fp2, F3, F4) and possibly insular cortex (electrodes C5, C6).

According to some embodiments, the neuroplasticity and/or excitability of the neuro structures in the specific brain regions may be evaluated by providing a diagnostic stimulation to the specific brain regions, and measuring the neural activity response of the neural structures in the targeted brain regions. According to some embodiments, a healthy neuroplasticity may be indicated by a change in the neural activity response over stimulations.

According to some embodiments, after evaluating the neuroplasticity and/or excitability of the neuro structures in the targeted brain region(s), a stimulation treatment to the brain region(s) may be provided. Such a stimulation treatment may affect the neuroplasticity and/or excitability of the neuro-structures, thereby providing a treatment to the chronic pain.

As used herein, the term “stimulation” refers to an intervention to the normal behavior/activity of the neurons/neuro-structures, and may be used for either inhibiting or stimulating a neural activity.

According to some embodiments, the stimulation is an electric stimulation. According to some embodiments, the stimulation is an electrically induced electric stimulation. According to some embodiments, the stimulation is a magnetically induced electric stimulation. According to some embodiments, the stimulation is a Transcranial-Magnetic-stimulation.

According to some embodiments, the stimulation includes inducing a magnetic flux/field to the brain or portions thereof. According to some embodiments, the magnetic stimulation may include a Transcranial-Magnetic-stimulation (TMS). According to some embodiments, the stimulation may include multiple TMS magnetic pulses at determined intensities. According to some embodiments, the TMS intensity may refer to the flux of the induced magnetic field. According to some embodiments, the TMS intensity may be measured/presented in Tesla units. According to some embodiments, the TMS intensity may be measured/presented as a percentage of a reference value.

According to some embodiments, measuring the reactive activity may be facilitated by measuring an electric/electromagnetic activity of neural structures in the brain. According to some embodiments, measuring the reactive activity may be facilitated by utilizing at least one electrode, configured to measure variations of electric/electromagnetic fields indicative of an activity of specific neural networks in the brain. According to some embodiments, measuring the reactive activity may be facilitated by utilizing an EEG device/system.

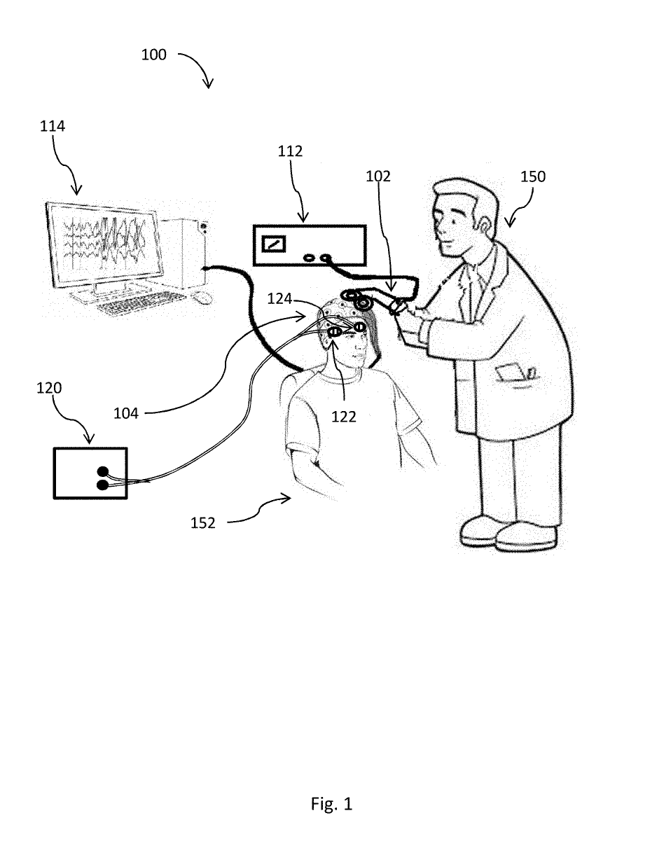

Reference is now made to FIG. 1, which schematically illustrates a setting 100 of treating chronic pain of a subject 152 by a care provider 150, according to some embodiments. As illustrated, care provider 150 places an evaluation stimulating device, such as but not restricted to TMS-inducer 102, at a certain position on/near the head of subject 152 for inducing stimulation to a target brain region thereof. TMS-inducer 102 is provided with control signals and electric energy from a controller, such as a TMS-controller 112. TMS-controller 112 is configurable for enabling a controllable stimulation by TMS-unit 112. According to some embodiments, a controllable stimulation may include control over the intensity, duration, frequency, flux and/or other stimulation-related parameters and patterns of stimulation.

At least some brain activity sensors, such as EEG-electrodes 104, are placed on the head of subject 152, and configured to measure neural activity of the brain of subject 152 or defined areas thereof for assessing a neural activity response to the diagnostic stimulation. According to some embodiments, EEG-electrodes 104 are in communicational link with an analyzer/controller, such as an EEG-analyzer 114, configured to obtain EEG signals from EEG-electrodes 104.

According to some embodiments, EEG-analyzer 114, or other processing circuitry (such as remote servers, a cloud computing service, a local computer, distributed computers, and the like), is configured to analyze the obtained EEG signals and detect reactive activity associated with induced electrical stimulation, such as TMS diagnostic stimulations induced by TMS-inducer 102. According to some embodiments, EEG-analyzer 114 is configured to facilitate evaluation of neuroplasticity and/or excitability of the targeted brain regions of subject 152 (or neural structures thereof) by measuring the intensities of the reactive activity, for example, by comparing the intensities with model/reference activities expected assuming various conditions and/or lack thereof. According to some embodiments, EEG-analyzer 114 is configured to detect neuroplasticity and/or excitability of the targeted brain regions of subject 152 (or neural structures thereof) by measuring the intensities of the reactive activity and comparing the ratios or differences between various pairs therefrom, and comparing the intensities with model/reference activities expected assuming various conditions and/or lack thereof.

According to some embodiments, EEG-analyzer 114 is configured to analyze the EEG signals relying on their amplitudes, slopes, frequencies, delays, area under curve and the like. According to some embodiments, EEG-analyzer 114 is configured to perform a frequency analysis of the EEG signals. According to some embodiments, EEG-analyzer 114 is configured to perform noise reduction filtration on the EEG-signals. According to some embodiments, EEG-analyzer 114 is configured to perform noise cancelation filtration on the EEG-signals. According to some embodiments, EEG-analyzer 114 is configured to perform a temporal analysis of the EEG signals.

According to some embodiments, a treatment stimulation mechanism is provided, such as transcranial current stimulation (tCS) device 120, which is configured to provide treatment brain stimulation to the target brain regions by driving an electric signal through a cathode 122 and an anode 124 placed on subject 152 at desired positions for inducing stimulation to the target brain regions.

According to some embodiments, a Neuronal stimulator, such as, TMS-unit 102 is configured to induce multiple stimuli pulses. According to some embodiments, the multiple stimuli pulses are substantively equal in intensity. According to some embodiments, the multiple stimuli pulses are substantively equal in duration. According to some embodiments, the multiple stimuli pulses are substantively similar in slopes. According to some embodiments, the multiple stimuli differ in intensity, duration, and/or slopes.

According to some embodiments, Transcranial current stimulation device 120 is a transcranial direct current stimulation device (tDCS). According to some embodiments, Transcranial current stimulation device 120 is a transcranial alternate current stimulation device (tACS).

According to some embodiments, the tACS is performed by inducing an alternating current at a frequency in the range of 1-120 Hz at current density of up to 5 A/m² to be matched with a desired oscillatory behavior needed, for example to induce 8-12 Hz activity, a 10 Hz tACS frequency will be administered.

Unbalanced plasticity between relevant cortical brain regions (such as but not limited to the “pain matrix”) will indicate chronic pain related changes, as well as plasticity shifts between visual and motor/sensory areas. Plasticity shifts in the prefrontal areas may also be associated with chronic pain.

According to some embodiments, treatment stimulation is configured to balance the plasticity between motor and/or sensory hemisphere. According to some embodiments, for short term plasticity changes, anodal stimulation may be applied in high plasticity areas to decrease the plasticity, and cathodal stimulation may be applied in low plasticity areas to increase local plasticity, for creating a balanced plasticity.

According to some embodiments, determination of pain related brain areas may be done by, measuring plasticity changes in pain related brain areas, and source localization of entire EEG data which determines the source brain region of EEG behavior. According to some embodiments, after establishing the brain areas to be treated, stimulation electrode positioning may be placed directly on these areas with supporting electrodes in contralateral and distant areas. The electrode locations may be calculated and optimized by electric flow modeling of a human average head with designated brain areas to be affected.

For example:

For right hand pain area to subject displaying high plasticity in left motor area and in left prefrontal area, a possible treatment electrode configuration may include anodal stimulation in C3 and Cp5 position, cathodal stimulation in C4 and Cp6 and anodal stimulation to Fp1 position. This treatment may decrease plasticity post stimulation in left motor area and increase plasticity in the contralateral area, while decreasing plasticity in left prefrontal area.

For left leg pain area to subject displaying low plasticity in right motor area and high plasticity in left prefrontal area, a possible treatment electrode configuration may include cathodal stimulation in C4 and Cp6 position, anodal stimulation in C3 and Cp5 and anodal stimulation to Fp1 position, for example. This treatment may increase plasticity post stimulation in right motor area and decrease plasticity in the contralateral area, while decreasing plasticity in left prefrontal area.

According to some embodiments, the devices, systems and/or methods may be used for assessing the progress and/or state of a chronic pain patient. According to some embodiments, the devices, systems and/or methods may be used for measuring a stage/severity of neural changed caused by chronic pain. According to some embodiments, the devices, systems and/or methods may be used for detecting and/or assessing neural network abnormalities.

According to some embodiments, evaluating the neuroplasticity of target brain regions may be done by providing stimulation to the target brain region and measuring the reactive neural activity of that region. According to some embodiments, evaluating the neuroplasticity of target brain regions may be done by iteratively providing stimulation to the target brain region, measuring the reactive neural activity of that region, and assessing the changes in reactive neural activity between different iterations.

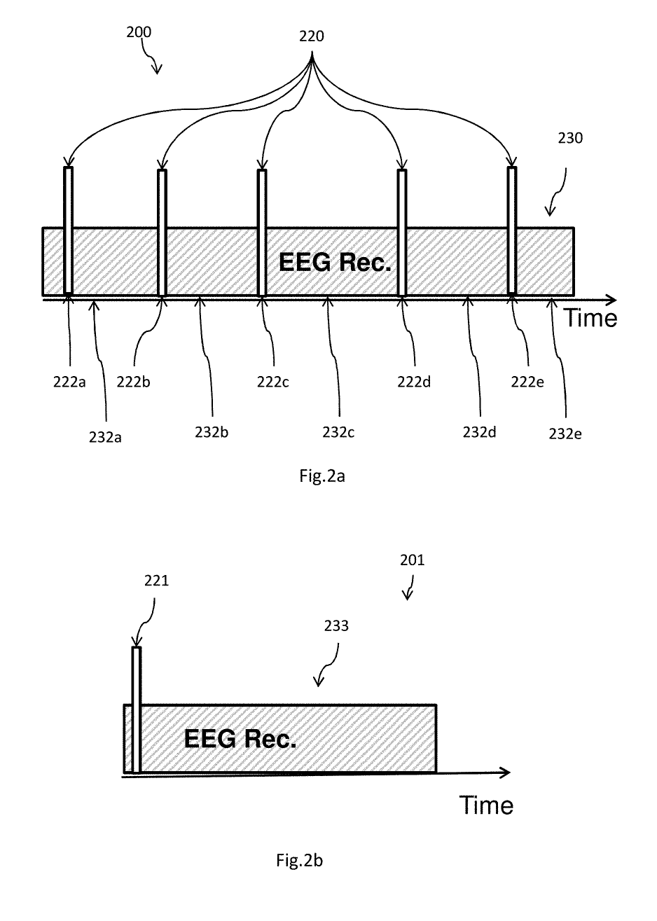

Reference is now made to FIG. 2a, which schematically illustrates a repetitive evaluation process 200, according to some embodiments. According to some embodiments, evaluation process 200 may include multiple diagnostic stimulation events, such as diagnostic stimuli pulses 220, configured to induce stimulation ,evoked by a diagnostic stimulation device, such as a TMS, at determined times and intensities. According to some embodiments, evaluation process 200 further includes sensing brain activity of the user, for example using EEG-monitoring 230 for measuring the response activity to stimuli pulses 220. According to some embodiments, EEG-monitoring 230 may be continuous throughout the period of stimulation burst 220. According to some embodiments, EEG monitoring 230 may be intermittent.

According to some embodiments, stimuli pulses 220 comprise a first stimulus pulse 222a followed by a first pulse delay interval 232a, a second stimulus pulse 222b followed by a second pulse delay interval 232b, a third stimulus pulse 222c followed by a third pulse delay interval 232c, a fourth stimulus pulse 222d followed by a fourth pulse delay interval 232d and a fifth stimulus pulse 222e followed by a fifth pulse delay interval 232e.

According to some embodiments, pulse delay intervals 232a, 232b, 232c, 232d, 232e are configured to enable distinguishing between EEG recording/monitoring of reaction activity associated with each of stimulus pulse 222a, 222b, 222c, 222d, 222e.

According to some embodiments, stimulus pulses 222a, 222b, 222c, 222d, 222e may be substantively equal in intensity. According to some embodiments, stimulus pulses 222a, 222b, 222c, 222d, 222e may vary in intensity.

According to some embodiments, stimulus pulses 222a, 222b, 222c, 222d, 222e may be substantively equal in duration. According to some embodiments, stimulus pulses 222a, 222b, 222c, 222d, 222e may vary in duration.

According to some embodiments, pulse delay intervals 232a, 232b, 232c, 232d, 232e may be substantively equal in duration. According to some embodiments, pulse delay intervals 232a, 232b, 232c, 232d, 232e may vary in duration.

Reference is now made to FIG. 2b, which schematically illustrates a singular evaluation process 201, according to some embodiments. According to some embodiments, an evaluation of the neuroplasticity of the target brain region may be enabled by a non-iterative stimulation event 221 and monitoring of a neural reactive response thereto, for example by EEG-monitoring 233.

According to some embodiments, the duration of a pulse is in the range of 0.5 ms to 2 ms. According to some embodiments, the duration of a pulse is in the range of 1 ms to 1.5 ms. According to some embodiments, the duration of a pulse is approximately 1 ms.

According to some embodiments, the delay intervals are in the range of 100 ms to 2 s. According to some embodiments, the delay intervals are in the range of 250 ms to 1 s. According to some embodiments, the delay intervals are approximately of 500 ms. According to some embodiments, the pulse frequency within a burst is in the range of 0.1 Hz to 10 Hz. According to some embodiments, the pulse frequency within a burst is in the range of 1 Hz to 5 Hz. According to some embodiments, the pulse frequency is approximately 2 Hz.

According to some embodiments, the reaction activity of the neural network in the brain is expected to vary between consecutive stimulus pulses. This may be attributed to the neuro-structural characters of plasticity and excitability. As a result, the reaction activity associated with the second stimuli pulse may be considerably higher than the reaction activity associated with the first stimuli pulse. Additionally, in later stimulus pulses, a gradual decrease in the reaction activity may be expected.

The amount/ratio of increased and/or decreased activity may be indicative of the characteristics of the neuronal network (neural structures), such as plasticity and excitation and inhibition. Consequently, one may detect irregularities in these characteristics and associate them with various conditions that may lead to changes in these characteristics.

According to some embodiments, a stimulation burst may include 1 or more stimuli pulses. According to some embodiments, a stimulation burst may include 2 or more stimuli pulses. According to some embodiments, a stimulation burst may include 3 or more stimuli pulses. According to some embodiments, a stimulation burst may include 4 or more stimuli pulses. According to some embodiments, a stimulation burst may include 5 or more stimuli pulses. According to some embodiments, a stimulation burst may include 2 to 10 stimuli pulses. According to some embodiments, a stimulation burst may include 10 or more stimuli pulses.

According to some embodiments, a session of evaluation and treatment is provided by performing evaluation (assessment of neuroplasticity) and providing treatment stimulation. According to some embodiments, an efficiency of a treatment may be evaluated by performing another evaluation (assessment of neuroplasticity), and based on the effectiveness assessment, another treatment session may be provided. According to some embodiments, parameters and characteristics of the second and following treatment sessions may be determined based on the assessed effectiveness of the first treatment.

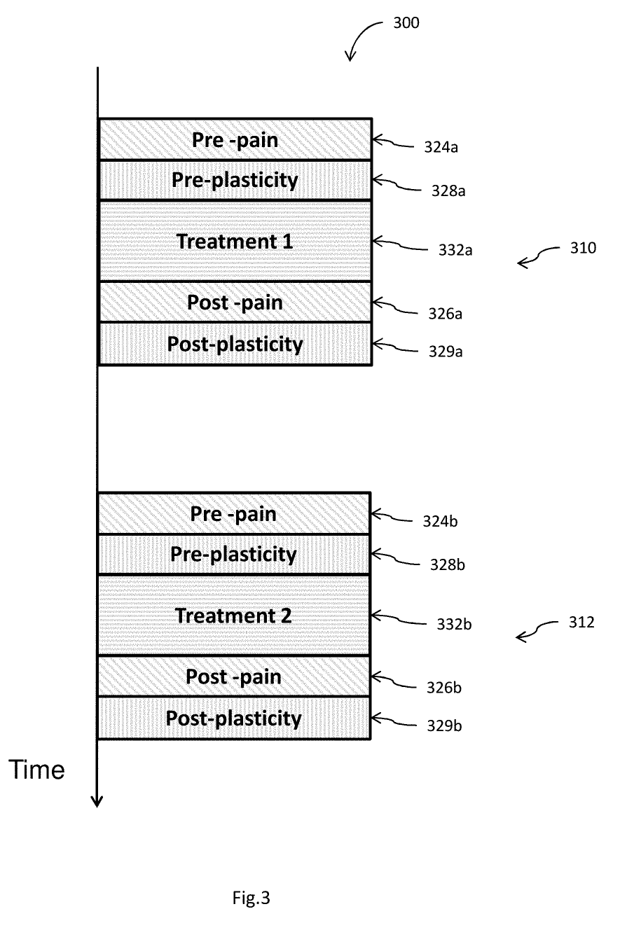

Reference is now made to FIG. 3, which schematically illustrates a session 300 of evaluation and treatment, according to some embodiments. According to some embodiments, the subject may undergo a first round of treatment 310, where he is asked to provide a score indicating a level of pain they feel and record a pre-treatment-pain 324a score, then the pre-treatment plasticity of certain brain regions may be evaluated 328a for determining a treatment, and a treatment stimulation 332a may be provided. Afterwards, the subject may be asked to provide a post treatment pain score 326a, and a post treatment plasticity is evaluated 329a to assess the first treatment efficacy.

According to some embodiments, the subject may then, after a period of time, undergo a second round of treatment 312, in which he is asked again to provide a score indicating a level of pain they feel and record a second pre-treatment-pain 324b score, then the plasticity of certain brain regions may be evaluated 328b for determining a treatment and progress compared to first round of treatment 310, and a second treatment stimulation 332b may be provided. Afterwards, the subject may be asked to provide a second post-treatment pain-score 326b, and a post treatment plasticity is evaluated 329b to assess the second treatment efficacy.

According to some embodiments, a stimulation session may include 2 or more evaluation periods. According to some embodiments, a stimulation session may include 3 or more evaluation periods. According to some embodiments, a stimulation session may include 5 to 10 evaluation periods. According to some embodiments, a stimulation session may include 10 or more evaluation periods.

According to some embodiments, an evaluation period may be essentially as described in FIG. 2a or FIG. 2b.

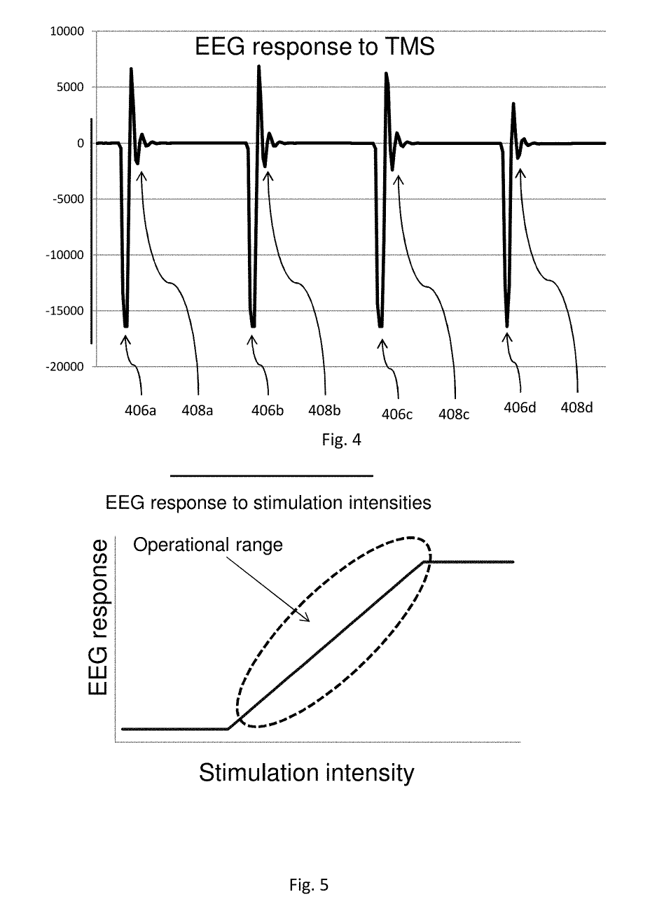

For assessing the plasticity and/or excitability of the predetermined brain region(s), TMS stimulation may be applied and the neural activity reaction may be monitored and analyzed. According to some embodiments, some parameters, characteristics and/or features that may be considered for assessing the neuroplasticity characteristics may include one or more of the following: amplitudes, slopes, frequencies, delays, area under curve and the ratios between. According to some embodiments, these parameters may be in response to stimulation pulses, or with association thereto. Reference is now made to FIG. 4, which schematically illustrates an EEG response activity to a stimulation burst in a normal brain, according to some embodiments. As illustrated, the stimulation burst includes four stimulation pulses; the EEG recording thereof is illustrated in a first pulse recording 406a, a second pulse recording 406b, a third pulse recording 406c and a fourth pulse recording 406d. Following each pulse recording, there is a response recording, namely a first response recording 408a, a second response recording 408b, a third response recording 408c and a fourth response recording 408d. As illustrated, second response recording 408b and third response recording 408c are greater than first response recording 408a, which may be attributed to the excitability or plasticity characteristics of the relevant neural network, while forth response recording 408d is considerably reduced compared to the rest of the responses recorded, which may be attributed to the adaptation characteristics of the relevant neural network.

According to some embodiments, responses that vary from the normal response or a normal response range may indicate various abnormal neural network characteristics.

According to some embodiments, the intensity of pulses is determined as a percentage of a reference value. According to some embodiments, the reference value is determined as a percentage from the personal determined motor-threshold value.

According to some embodiments, a motor threshold is a stimulation intensity at which a motoric reaction may be triggered and/or detected as a consequence thereto.

According to some embodiments, a motor threshold value is established by increasingly incrementing stimulation intensity, until a motoric reaction is detected. According to some embodiments, the motoric threshold may vary from one person to another. According to some embodiments, using a TMS stimulator to induce focal electrical stimulation in the brain, the motor threshold value may be in the range of 1.5 to 2.5 Tesla. According to some embodiments, the motor threshold value may be in the range of 1.7 to 2.3 Tesla. According to some embodiments, the motor threshold value may be in the range of 1.8 to 2.2 Tesla. According to some embodiments, the motor threshold value may be in the range of 1.9 to 2.1 Tesla. According to some embodiments, the motor threshold value may be in the range of 48% to 52% of the maximal TMS device intensity. According to some embodiments, the motor threshold value may be approximately 50% of the maximal TMS device intensity.

Once a motor threshold value is established for a certain person, one may evaluate/calculate an “operational range”, which is a range of intensity values in which a reaction activity may be observed and activity corresponds to the intensity of the stimuli.

According to some embodiments, the term “operational range” may be interchangeable with the terms “relevant range” or “active range”.

According to some embodiments, an “operational range” ranges from 60% to 140% of the motor threshold. According to some embodiments, an “operational range” ranges from 80% to 120% of the motor threshold. According to some embodiments, an “operational range” ranges from 50% to 150% of the motor threshold.

According to some embodiments, the intensity of the stimuli pulses may be a value within the “operational range”. According to some embodiments, the intensity of the stimuli pulses may be referred to as an “operational point”.

Reference is now made to FIG. 5, which schematically illustrates a stimulation intensity operational range, according to some embodiments. As illustrated, the EEG response to low stimulation intensities is relatively unchanged, until the stimulation intensity surpasses a certain value (lower threshold), then the EEG response reacts/increases as the stimulation intensity increases, until the stimulation intensity reaches another value (upper threshold) in which the EEG response no longer responds to increases in the stimulation intensity.

According to some embodiments, the operational range is a range of intensity values between the lower threshold and the upper threshold.

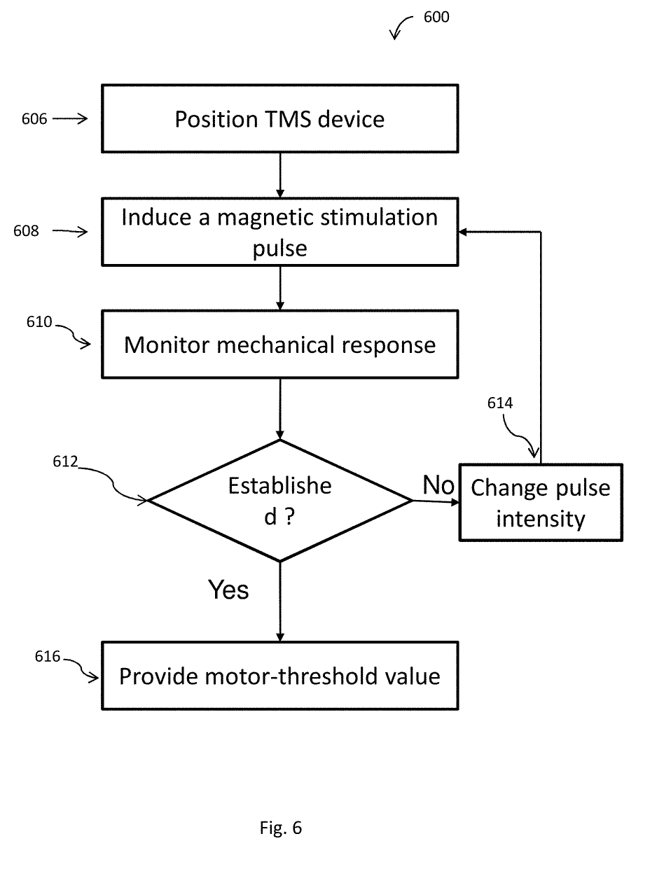

Reference is now made to FIG. 6, which schematically illustrates a method 600 for establishing a motor threshold value, according to some embodiments. According to some embodiments, the TMS device/unit is positioned (step 606) in a determined position on/near the head of the subject, then a magnetic stimulation pulse is induced (step 608), then the mechanical response is monitored (step 610) for detecting triggered motoric movement. According to some embodiments, if a motoric movement is detected, and a motor threshold established (step 612), the motor threshold value is provided (step 616), otherwise, the pulse intensity is changed (step 614) and we iterate the steps from inducing the magnetic stimulation pulse (step 608).

According to some embodiments, changes to the pulse intensity (step 614) include increasing the pulse intensity.

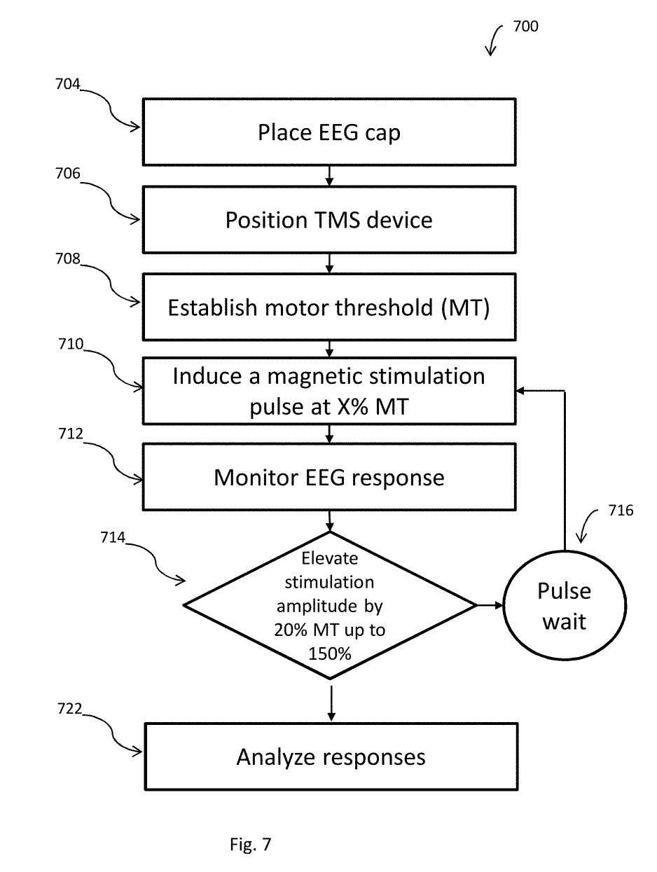

Reference is now made to FIG. 7, which schematically illustrates a method 700 for providing a stimulated EEG response session, according to some embodiments. According to some embodiments, an EEG cap is placed on the head of a subject (step 704), then a TMS device is positioned on/near the head of the subject (step 706), then a motor threshold (“MT”) is established (step 708), then a magnetic stimulation pulse is induced (step 710) having an intensity of X % of the established MT. According to some embodiments, the X % is a predetermined percentage of the MT. According to an operational range/value based on the motor threshold, then the EEG response activity is monitored, (step 712), then optionally steps 710 and 712 are repeated as the stimulation amplitude is elevated by a predetermined percentage up to a certain level, for example 20% up to 150%, (step 714) with a “pulse-wait” delay between iterations (step 716), and the response activity is analyzed (step 722).

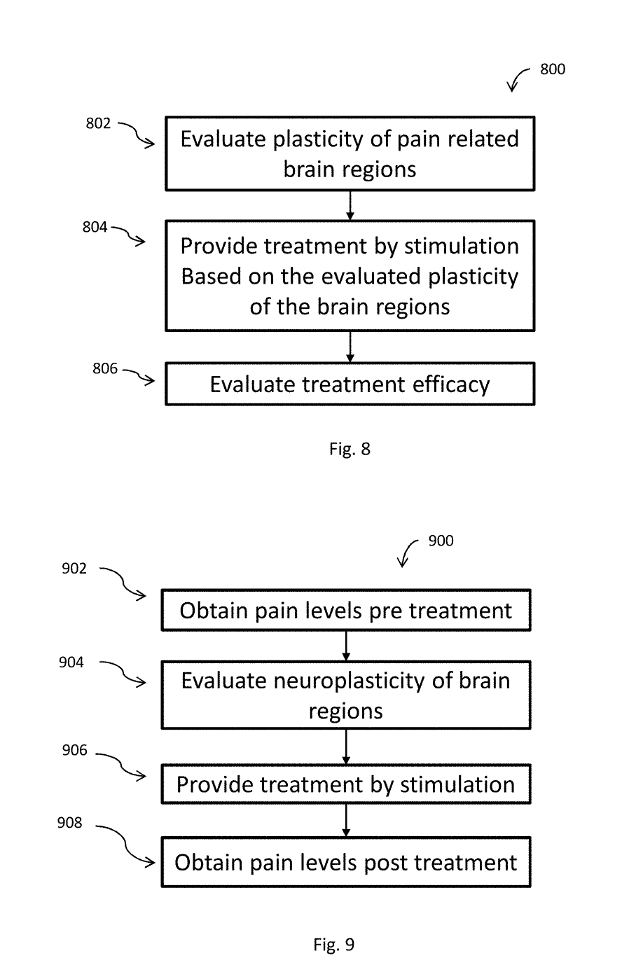

Reference is now made to FIG. 8, which schematically illustrates a high-level method 800 for treating chronic pain, according to some embodiments. According to some embodiments, method 800 begins by an evaluation of the plasticity of pain-related brain regions (step 802), then a treatment by stimulation based on the evaluated plasticity of the brain regions (step 804) then an evaluation for the treatment efficacy (step 806), for example by measuring the plasticity once again.

According to some embodiments, the subjects may be asked to evaluate the pain level they are feeling, and the efficacy of the treatment may be measured by the changes in the pain levels the subject is experiencing.

Reference is now made to FIG. 9, which schematically illustrates a method 900 for treating chronic pain, according to some embodiments. According to some embodiments, method 900 begins with obtaining pain levels that the subject is suffering/feeling before the treatment (step 902), then evaluate the neuroplasticity of pain related brain regions (step 904), then a stimulation treatment is provided to the pain related brain regions (step 906), and the pain levels that the user feels after the treatment may be obtained (step 908) to check whether the treatment was successful.

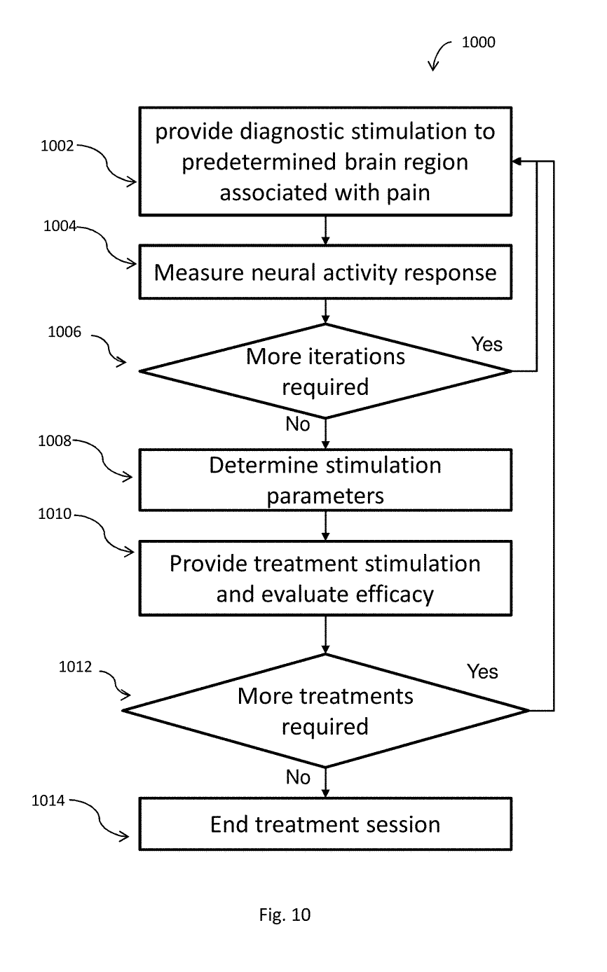

Reference is now made to FIG. 10, which schematically illustrates a method 1000 for iterative evaluation and treatment, according to some embodiments. According to some embodiments, method 1000 begins by providing a diagnostic/examinatorial stimulation to a predetermined brain region (step 1002) associated with pain, then measuring neural activity response to the diagnostic/examinatorial stimulation (step 1004), and if more diagnosis/evaluation iterations are required (step 1006), go back to providing a diagnostic/examinatorial stimulation to a predetermined brain region (step 1002), otherwise, proceed by evaluating the neuroplasticity and determine stimulation parameters accordingly (step 1008) and provide a treatment stimulation and evaluate the efficacy thereof (step 1010), then if more treatments may be required (step 1012), go back to providing a diagnostic/examinatorial stimulation to a predetermined brain region (step 1002), otherwise, end treatment session(step 1014).

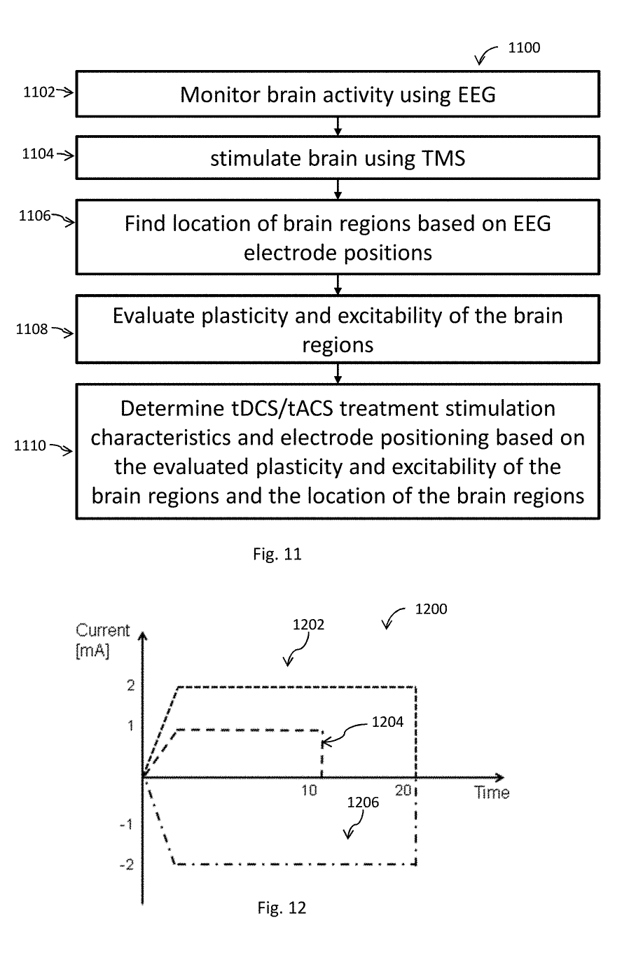

Reference is now made to FIG. 11, which schematically illustrates a method 1100 for treating chronic pain with tCS stimulation, according to some embodiments. According to some embodiments, the brain activity is monitored by an EEG system (step 1102), then the brain is stimulated using a TMS machine (step 1104) and the neural reaction response is analyzed for finding the location of the brain regions based on the EEG electrode positions (step 1106), and the neuroplasticity and excitability of the brain regions is evaluated (step 1108), based on the neuroplasticity and the brain region locations, a tDCS/tACS treatment stimulation is configured with characteristics and montage (electrode placements) (step 1110) for providing a personalized pain treatment stimulation.

Reference is now made to FIG. 12, which schematically illustrates tDCS sessions 1200, according to some embodiments. According to some embodiments, various tDCS parameters may be determined based on the stimulation need, for example strong-anodal-stimulation 1202 may be configured to provide a ramp-up from 0 mA to 2 mA, and drive 2 mA current for 20 minutes and end the stimulation. According to some embodiments, a weak-anodal-stimulation 1204 may be configured to provide a ramp-up from 0 mA to 1 mA, and drive 1 mA current for 10 minutes and then end stimulation. According to some embodiments, strong-kathodal-stimulation 1206 may be configured to provide a ramp-up from 0 mA to −2 mA, and drive −2 mA current for 20 minutes. According to some embodiments, the duration, current value and current direction may be configurable.

According to some embodiments, anodal stimulation may stimulate the neural activity of a brain region, while a cathodal stimulation may inhibit the activity of the brain region, depending on the electrode locations. According to some embodiments, the electrode polarity is selected (anodal/cathodal/alternate), an electrode montage (number of cathodes and anodes), and location, for example over M1 or S1 or Dorsolateral prefrontal cortex (DLPFC) brain regions, all corresponding to painful area.

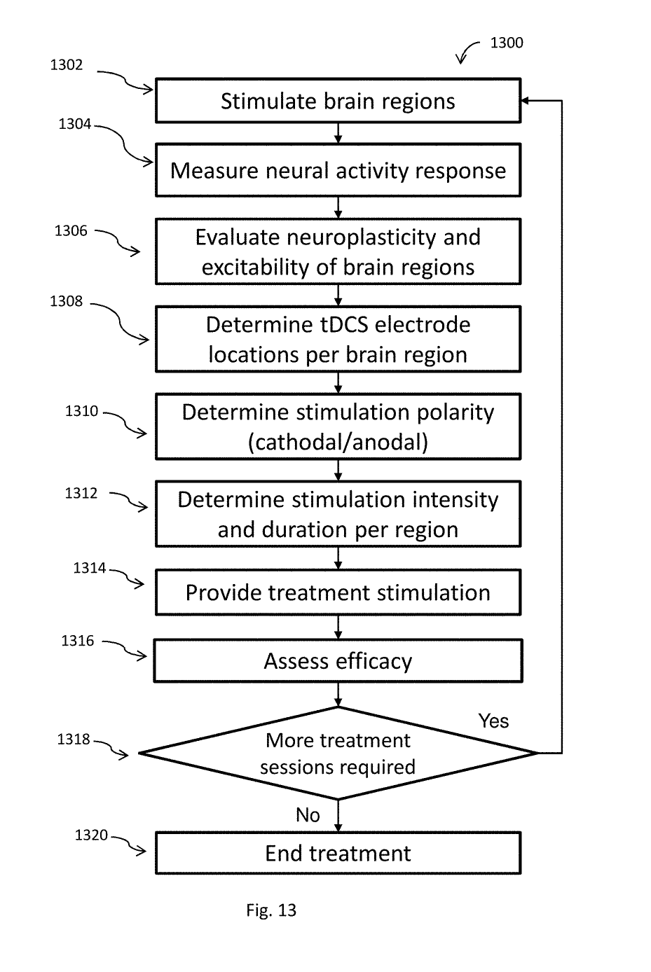

Reference is now made to FIG. 13, which schematically illustrates a method 1300 for treating chronic pain with tDCS stimulation, according to some embodiments. According to some embodiments, method 1300 begins with stimulating pain-related brain regions (step 1302), and measuring the neural activity response (step 1304) to evaluate the plasticity and excitability of the brain regions (step 1306), then determining tDCS electrode location per brain region (step 1308), determine stimulation polarity per brain region (step 1310) and determine stimulation intensity and duration (step 1312), and provide the direct current stimulation according to the above determined parameters (step 1314), then assess the efficacy of the treatment (step 1316), and decide whether more treatment sessions are required (step 1318). If more sessions are requires, we go back to stimulating the brain regions (step 1302), if not, we end the treatment (step 1320).

According to some embodiments, the parameters/characteristics of a stimulation session may be determined, taking into consideration previous sessions and the efficacy thereof.

According to some embodiments, a stimulation threshold value is established. According to some embodiments, a stimulation threshold value is a TMS stimulation intensity value, above-which reactive neural activity may be observed/detected.

According to some embodiments, the TMS stimulation intensity is a “supra-threshold”, selected to be of a higher intensity than the stimulation threshold.

According to some embodiments, the TMS stimulation intensity is a “sub-threshold”, selected to be of a lower intensity than the stimulation threshold.

According to some embodiments, the analysis is conducted after the stimulation session. According to some embodiments, the analysis is conducted during the stimulation session.

As used herein, the terms “neuron”, “neuro-structure”, “neural network” may be interchangeable.

As used herein, the term “TMS” may refer to Transcranial magnetic stimulation, which is a non-invasive method used to stimulate regions of the brain. In TMS, a magnetic field generator, such as a coil or an electromagnet, is placed near/on the head of the subject receiving the stimulation, an electric current is conducted through the coil and a magnetic flux gradient is induced as a result of the change in current through the coil. According to some embodiments, the coil (or electromagnet coil) is connected to a pulse generator, or controller, or stimulator configured to deliver electric current to the coil.

As used herein, the terms “tCS”, “tDCS” and “tACS” refer to a brain stimulation technique in which electric current is driven though the crania of the user to reach and stimulate a desired region, and the electric current is provided to the crania using at least two electrodes placed at defined places such that driving a current signal via the electrodes through the crania may reach the desired brain region.

As used herein, the term “EEG” may refer to Electroencephalography, which is typically a non-invasive method for recording electrical activity of the brain along the scalp. An EEG measures voltage fluctuation resulting from ionic current within the neurons/neuro-structures of the brain. According to some embodiments, an EEG may refer to the recording of the brain's spontaneous and/or stimulated electrical activity over a period of time.

As used herein, the term “plasticity” may refer to neuroplasticity or brain plasticity which may encompass synaptic and/or non-synaptic plasticity, and may refer to changes in neural pathways and synapses and/or structure due to changes in behavior, environment, neural processes, thinking, emotions, injuries and stimulation.

As used herein, the terms “examinatorial” and “diagnostic” stimulation may be interchangeably used and refer to stimulation(s) that are intended to examine a neural behavior and/or diagnosing a cognitive condition.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A method for treating chronic pain of a subject, the method comprising:
inducing a diagnostic stimulation to a predetermined brain region of the subject;
measuring a neural activity response to the diagnostic stimulation;
evaluating a neuroplasticity and/or excitability of neural-structures in the predetermined brain regions based on the neural activity response to the diagnostic stimulation; and
inducing a treatment stimulation to the predetermined brain region based on the evaluated neuroplasticity of the neural structures in the predetermined brain region.

2. The method of claim 1, wherein the chronic pain is a neuropathic pain.

3. The method of claim 1, wherein the predetermined brain region is selected from a group comprising: primary motor cortex and primary somatosensory cortex, prefrontal cortex and insular cortex.

4. The method of claim 1, wherein inducing a diagnostic stimulation to a predetermined brain region of the subject comprises utilizing transcranial magnetic stimulation (TMS).

5. The method of claim 1, wherein inducing a diagnostic stimulation to a predetermined brain region of the subject comprises providing the subject with a cognitive/behavioral task configured to induce stimulation to the predetermined brain region.

6. The method of claim 1, wherein measuring a neural activity response to the diagnostic stimulation comprises obtaining electroencephalogram (EEG) measurements indicative of a neural activity of the brain of the user during and/or after inducing the diagnostic stimulation.

7. The method of claim 6, wherein inducing a treatment stimulation to the predetermined brain region comprises electric brain stimulation.

8. The method of claim 7, wherein the inducing electric brain stimulation comprises placing a first electrode and a second electrode on the subject, such that an electric signal passing through the user via the first electrode and the second electrode reaches a predetermined brain region.

9. The method of claim 7, further comprising evaluating specific brain areas for plasticity changes by dipole fitting the EEG measurements and performing a source modeling for detecting the location (source localized areas) of the specific areas and providing treatment stimulation to the source localized areas.

10. The method of claim 7, wherein the electric brain stimulation comprises transcranial direct current stimulation (tDCS), or transcranial alternating current stimulation (tACS).

11. The method of claim 10, wherein the tDCS utilizes a current density of no more than 5 A/m2, and has a duration of no more than 30 minutes.

12. The method of claim 10, wherein the tDCS stimulation is selected to be either Anodal stimulation or Cathodal stimulation for a brain region based on the evaluated neuroplasticity and/or excitability of neural-structures in the predetermined brain regions.

13. (canceled)

14. The method of claim 10, wherein the tACS is performed by inducing an alternating current at a frequency in the range of 1-120 Hz at current density of 5 A/m2 to be matched with a desired oscillatory behavior needed.

15. (canceled)

16. (canceled)

17. The method of claim 1, further comprising:
inducing a post-treatment diagnostic stimulation to the predetermined brain region of the subject;
measuring a neural activity response to the post-treatment diagnostic stimulation;
evaluating a post-treatment neuroplasticity and/or excitability of neural- structures in the predetermined brain regions based on the neural activity response to the post-treatment diagnostic stimulation; and
inducing a treatment stimulation to the predetermined brain region based on the evaluated post-treatment neuroplasticity of the neural structures in the predetermined brain region.

18. A system for treating chronic pain of a subject, the system comprising:
a directed inspective/diagnostic stimulation unit, configured to induce a diagnostic stimulation to a predetermined brain region of the subject;
a brain activity sensor, configured to measure a neural activity response to the diagnostic stimulation induced by said directed brain stimulation unit;
a processing circuitry in communication with said brain activity sensor, said processing circuitry is configured to evaluate a neuroplasticity and/or excitability of neural-structures in the predetermined brain region based on the neural activity response to the diagnostic stimulation; and an electric current stimulation unit comprising a first electrode and a second electrode, said electric current stimulation unit is configured to induce a treatment stimulation to the predetermined brain region based on the evaluated neuroplasticity of the neural structures in the predetermined brain region, by driving a current signal through the user via the first electrode and the second electrode such that the current reaches the predetermined brain region.

19. The system of claim 18, wherein said directed inspective stimulation unit comprises a transcranial magnetic stimulation (TMS) unit.

20. The system of claim 18, wherein said brain activity sensor comprises an electroencephalography (EEG) device.

21. The system of claim 20, wherein said processing circuitry is configured to obtain an EEG signal from said EEG device and evaluate a neuroplasticity and/or excitability of neural-structures in the predetermined brain region by comparing the neural activity response to the diagnostic stimulation with previously obtained neural activity response to previous diagnostic stimulation.

22. The system of claim 20, wherein said processing circuitry is further configured to estimate locations of the predetermined brain regions based on the obtained EEG signal.

23. The system of claim 18, wherein said electric current stimulation unit comprises a transcranial direct current stimulation (tDCS) device subject or a transcranial alternate current stimulation (tACS) device.

24. (canceled)
